(19) United States
(12) Patent Application Publication
(10) Pub. No.: US 2009/0258088 A1
(43) Pub. Date: Oct. 15, 2009

(54) USE OF MATERIALS AND EXTERNAL STIMULI FOR SYNOVECTOMY

(75) Inventors: R. Keith Frank, Lake Jackson, TX (US); Kenneth McMillan, Richwood, TX (US); Jaime Simon, Angleton, TX (US); Alan D. Strickland, Lake Jackson, TX (US)

Correspondence Address: Karen L. Kimble;Technology Law, PLLC, 3595 N. Sunset Way, Sanford, MI, 48657, US

(73) Assignee: Iso Therapeutics Group LLC, Angleton, TX (US)

(21) Appl. No.: 12/311,949

(22) PCT Filed: Oct. 24, 2007
(86) PCT No.: PCT/US07/22536

§ 371 Date: Apr. 20, 2009

Related U.S. Application Data
(60) Provisional application No. 60/853,919, filed on Oct. 24, 2006.

(51) Int. Cl. A61K 33/26; A61K 9/14; A61K 33/24

(52) U.S. Cl. 424/646; 424/489; 424/490; 424/617

(57) ABSTRACT

This invention has as one of its aspects a method to provide a safe an effective treatment for synovectomy. The method involves the administration a material to the affected joint, allowing the material to localize in the synovial membrane, and then applying an external stimulus that interacts with the material to provide therapy. The materials of this invention are preferably particles, which are either magnetic or contain a heavy element. The external stimuli of this invention includes an alternating magnetic field to heat magnetic particles, infrared laser to heat heavy elements, or electromagnetic ionizing radiation (X- or gamma-radiation) that interacts with heavy elements to produce a localized radiation dose.

### FIELD OF THE INVENTION

The present invention relates to the use of particles that concentrate in the synovium of a diseased joint followed by an external stimulus that interacts with the particles to cause synovectomy.

### BACKGROUND OF THE INVENTION

The synovial membrane, or synovium, is a thin layer of cells and fibrous tissue which covers most bone surfaces within joints. It does not generally cover cartilaginous surfaces of joints and may leave some bare bone surfaces in some joints. Synovium is histologically noted to have a continuum of Type A cells, which have the appearance of macrophages with abundant vacuoles, membrane invaginations, and cellular inclusions, to the Type B cells, which have prominent endoplasmic reticulum and produce the synovial fluid that fills the joint space. The synovium varies in depth from about 4 cells in thicker places to areas with very sparse cells, particularly in areas of pressure and over tendons or ligaments. Type B cells usually predominate in the synovium. However, under conditions such as joint infection, intra-articular hemorrhage, meniscal tears, autoimmune damage, and other diseases, the synovial cells may revert to the Type A cells with enhanced removal of intra-articular debris. In these circumstances, the synovium may hypertrophy and fill excessive space in the joint. Alterations in the composition of the synovial fluid may occur resulting in imbalances between collagen and glycoproteins such as fibronectin and laminin and the enzymes that remove these proteins. Joint damage may ensue from the secretory imbalances, the macrophage response, pressure changes from cellular hypertrophy, and other consequences of the switch to Type A cells.

### Chronic Inflammation—Synovitis.

The treatment of joints that have chronic inflammation is called synovitis. There are multiple causes of this condition including several types of arthritis and in particular, inflammatory arthritis. Mild cases can often be treated and controlled with existing drugs. However, severe chronic synovitis can lead to significant pain and joint damage. Present treatments for this condition include surgical synovectomy or joint replacement.

Psoriatic Arthritis. Psoriatic arthritis is considered an autoimmune disease where the patient's immune system reacts with the patient's normal tissues. It is characterized by inflammation of joints and is frequently associated with scaly, dry, and thick skin. The symptoms include swelling of the joints of the hands, feet, knees, and ankles. Inflammation can occur in only a few joints at a time and can become painful, swollen, hot and red. Joint stiffness and pain in the lower back, neck, and buttocks is also common. Psoriatic arthritis affects patients with psoriasis. It has been stated by the National Psoriasis Foundation that 2.2% of American adults have been diagnosed with psoriasis. They also state that 11% of those diagnosed with psoriasis have also been diagnosed with psoriatic arthritis. This is a prevalence of 0.25% of American adults in the general population. The Mayo Clinic estimates up to one million people in the United States have psoriatic arthritis (0.33%). Men and women are equally susceptible to the disease and the onset is usually between the ages of 20 and 50.

The cause of psoriatic arthritis is not known. Genetic factors and abnormalities in the immune system are thought to play a role. In addition, there may be environmental factors such as bacteria or fungal agents that are related to developing the disease. Most people can live normal lives with this disease; however about 20% of the population affected with psoriatic arthritis will have joints that will become deformed. The damage to the joint is caused by persistent inflammation of the membrane lining the joint called the synovium.

For less severe cases of psoriatic arthritis, anti-inflammatory drugs, exercise, rest and other treatments can be used to successfully manage the patient. However when early treatment options are not successful, more severe treatment options are used including joint replacement; and short of that, surgical removal of the inflamed lining tissue (synovium) from inside the joint. This type of surgery is called synovectomy.

Rheumatoid Arthritis. Rheumatoid arthritis (RA) is another autoimmune disease causing synovitis. Inflammation in the joint can lead to stiffness, pain and deformation of the joint. The exact cause of RA is not known, but it is suspected that it may be triggered by infections. It is estimated that 1% of the American population is afflicted with rheumatoid arthritis. This accounts for about 3 million cases of the disease in 2007. It is 2 to 3 times more prevalent in women than in men. Although RA can be present at any age, most patients are first diagnosed at 30-50 years of age. The disease typically begins with the slow development of signs and symptoms over weeks to months. Stiffness in joints is the first sign followed by pain and tenderness. In almost every case there is involvement of several joints—5 or more joints is common. The joints most commonly affected include joints of the hands, wrists, shoulders, elbows, knees and ankles.

Treatment options include nonsteroidal anti-inflammatory drugs [(commonly called NSAIDs) that reduce inflammation], analgesics (that relieve pain), glucocorticoids or prednisone (that slow joint damage), disease modifying antirheumatic drugs [also known as DMARDs (that slow joint destruction)], biologic response modifiers (reduce inflammation by inhibition of cytokines), and protein-A (used to filter the blood to remove antibodies and immune complexes). If medications, exercise and physical therapy fail to alleviate the pain, surgical synovectomy is an option.

### Intra-articular Hemorrhage.

The treatment of recurrent intra-articular hemorrhage, such as that which occurs in hemophilia is another area of concern for joint damage. In these cases, blood in the joints leads to a reversion to Type A synovial cells to remove the blood from the joint. When this recurs frequently, the Type A (macrophage-like) cells recruit an inflammatory response which can cause capillaries in the joint to leak, resulting in more episodes of joint hemorrhage. Synovectomy may be needed for these patients. Surgical synovectomy has been the usual method of treatment.

Other non-limiting diseases which may require synovectomy include synovial chondromatosis, pigmented villonodular synovitis, lipomas, and other tumors.

Surgical synovectomy suffers from the possibility of one or more of: a) a reaction to the anesthesia, b) the possibilities of blood clots, c) possible damage to ligaments or tendons associated with the joint, d) prolonged, post-surgical pain, and e) bone surface damage. Since it is not possible to surgically remove all the affected tissue, the synovium may grow back with the disease reoccurring at that joint.

For these reasons, radiation synovectomy has been done. This involves the introduction of a radioisotope into the joint that deposits enough energy in the synovium, as the radioisotope decays, to ablate the tissue. In most cases beta-emitting radionuclides, contained in particles, are used wherein the particles are absorbed to the membrane and the radiation emitted is enough to destroy the tissue. This approach has been used in the literature as seen from: Deutch, et al. (WO9105570 A1) that teach the use of Re-188 or Re-186 attached to albumin microspheres, sulfur colloids, or glass beads; Simon, et al. that teach the use of rare earth isotopes such as Sm-153, Ho-166, Y-90, and Lu-177 adsorbed on a previously prepared iron hydroxide particle (WO9211032 A1); and Brodack, et al. that teach the use of paramagnetic particles containing therapeutic radionuclides (WO9701304 A1). Clinical synovectomy using Au-198 has been reported over many years (e.g., Ortonowski, Ziemski, Kucharski, Woy-Wojciechowski in Folia Haematol Int Mag Kin Morph Blutforsch. 1990; 117(4): 505-510 and Petersson, C J. Haemophilia 2001; 7 Supl 2:31-33). The problem with radiation synovectomy is that after administration of radioactive particles in the synovium, the particles can migrate and irradiate other parts of the body. This is especially true if the synovium is compromised such that leakage is more of a problem.

O'Neal et al. (Cancer Letters. 2004 Jun. 25; 209(2): 171-6) describe the accumulation of a gold nanoparticle in tumors and treating the cancer by heating the particles with a near-infrared laser. The particles consist of a dielectric core such as silicone and a thin layer of metal such as gold. These particles are known to concentrate in tumors of animals and can be heated by exposing them to infrared light. This approach has shown promise in treating cancer; however the approach has not been applied to synovectomy.

Funke, et al. (Lasers Surg. Med. 2006 Sep. 14) describe the use of transdermal photodynamic therapy in a fibroblast-induced model of joint destruction using the photosensitizer tetrahydroporphyrin-tetratosylat. They propose this as a strategy for synovectomy; however their experiments resulted in skeletal muscle necrosis.

Ivkov, et al. (US200550090732 A1) teach the use of a magnetic nanoparticle in combination with an alternating magnetic field to heat tissues where the magnetic particles concentrate.

Gordon (U.S. Pat. No. 4,994,014) teaches the treatment of diseased cells by the intravenous injection of particles, allowing the particles to be intracellularly absorbed by diseased cells, and raising the oxygen level in the subject's blood. This increases the rate of the intracellular absorption of oxygen by the diseased cells. This oxygen absorption, together with the intracellularly-absorbed particles, then increases the rate of oxidation and metabolism of the particles, and thereby raises the subject's intracellular energy. The intracellular production of interleukins and other activators, such as interferons and prostaglandins, is thereby stimulated. These interleukins and other activators destroy the diseased cells wherever they may be in the subject. This process can be enhanced by then applying to the subject an alternating electromagnetic field tuned to a resonant frequency.

Cash, et al. (WO0012006 A1) teach the pre-treating of a tumor with heavy element materials followed by treating the area with external radiation. The heavy element enhances the radiation dose at the site where the heavy elements are deposited.

All four of these techniques circumvent the problem of radioactivity in the target tissue by introducing non-radioactive material to the tissue prior to treating with an external stimulus to provide the therapy. However, none of these references teach the use of this technique for ablating the synovium of patients afflicted with synovitis.

Neutron capture therapy to cause synovectomy has been previously described. See Watxon-Clark, et al., Proceedings of the National Academy of Sciences of the United States of America, 95 (5), 2531 (March 1988). This method involves the administration of either a boron or gadolinium containing compound into the joint that is taken up by the synovium. The joint is then subjected to a neutron field. The interaction of neutrons with boron or gadolinium results in the release of an alpha particle. This approach suffers from the complexity of delivering neutrons to a patient. Additionally the alpha particles created do not travel very far. Thus, to affect significant cell kill, the alpha particles need to be generated very close to the nucleous of the cell. In addition, neutrons can activate other elements creating radioactivity elsewhere in the body.

It is well recognized that materials such as particles can be readily incorporated into cells by the process of phagocytosis. A wide range of particle sizes may be utilized. Gade, et al. (Blood, 104(4), 916, 2004) successfully incorporated superparamagnetic iron particles from 17 to 900 nm into cells to study MR based cellular imaging. Polystyrene particles from 0.5 to 3 μm were used by Oh et al. (J. Cell Biology, 132, 585, 1996) to examine the fate of phagocytosed particles. Foged et al. (Int. J. Pharmaceutics, 298(2), 315, 2005) studied the phagocytosis by human dendritic cells of particles ranging from 0.04 μm (40 nm) to 15 μm and found that most efficient phagocytosis was with particle diameters less than 0.5 μm. She also found that the uptake of particles was enhanced by a positive surface charge.

### SUMMARY OF THE INVENTION

One aspect of this invention is to develop a safer and more efficacious therapeutic approach to synovectomy (ablation of the synovial membrane) in diseased joints without the use of surgery. This invention includes delivering a material to the synovial membrane prior to administering an external stimulus that provides the therapy. More specifically, this invention concerns a method of treatment of at least one synovial membrane of an affected joint in a patient having synovitis comprising:

(a) administering to the affected joint an effective amount of a material that becomes associated with the synovial membrane;

(b) allowing the material to localize and/or concentrate in the synovial membrane; and

(c) applying an external stimulus to the affected joint which interacts with the material thus delivering a therapeutic dose to ablate the synovial membrane.

When these materials are magnetic materials and they are delivered to the synovium, then the external stimulus is an alternating magnetic field that heats the particles and thermally ablates the synovium. When these materials are heavy element materials and they are delivered to the synovium, then the external stimulus can be either gamma rays or X-rays that interact with the heavy element providing a high radiation dose specifically at the synovium, thus accomplishing the therapy. Additionally, lasers such as near IR lasers can penetrate and cause local heating at the site of the heavy metal.

### DETAILED DESCRIPTION OF THE INVENTION

This invention provides a safe and efficacious method to deliver ablative treatment to the synovial membrane of a diseased joint. This is done by administering a material into the joint space of an affected joint that accumulates in the synovial membrane, followed by applying an external stimulus that interacts with the material. The preferred material of this invention is composed of particles.

While not wishing to be bound by theory, the mechanism of action for this invention is believed to be as follows. Since diseased synovium has switched to a preponderance of the Type A cells, the macrophage-like synovial cells will preferentially phagocytize these particles. While the synovial cells with the intracellular particles are still present in the synovium, an external stimulus is used to activate the particles and destroy the cells. This treatment preferentially destroys the synovial cells that are in the area of most active disease since these cells will be most active in phagocytosis.

The external stimulus is chosen such that it interacts with the administered material. For example, if a magnetic material is administered to the synovium, the external stimulus can be a magnetic field that can interact with the material to generate heat and provide a therapeutic effect specifically to the synovium. If a heavy element is administered to the synovium, the external stimulus can be low-energy photons such as gamma rays or X-rays which interact with the heavy element to provide ionizing radiation specifically at the synovium. Additionally, near-IR laser can penetrate into the joint and interact with heavy element material to cause heating. Any of these techniques may be used to provide a therapeutic dose to ablate the synovium.

The choices of administered material are varied. It is advantageous that there is a large quantity of material delivered to the synovium. Thus the preferred administered compounds are particulate materials such as particles that can be readily taken up (phagocytized) by the synovium. The preferred size of the particles is between 15 nanometers (nm) and 15 microns (μm); preferred is between 25 nm to 5 μm; most preferred is between 50 nm and 3 μm. These particles may comprise the material alone or in combination with other compounds that serve as carriers for the material and/or a targeting group such as a monoclonal antibody and/or the particles can have a surface coating, especially a cationic surface coating at physiological pH. Thus these particles may be administered as pharmaceutically-acceptable formulations wherein various chelating agents may be present or other solubilizing or suspending agents (e.g., saline, water for injection, buffers, etc.) to ensure the ease of administration of the particles to the synovium and other pharmaceutically-acceptable excipients or carriers, diluents, or anti-bacterial agents or preservatives may be present.

Magnetic Particles. The characteristics of the magnetic particles of this invention are those that can be heated with an external alternating magnetic field. For the magnetic particles of this invention, iron oxide or hydroxide particles are preferred.

Heavy Element Particles. The nature of the particles that they contain heavy elements is such that they contain element(s) of atomic number higher than 37. More preferred are particles that contain an element of atomic number greater than 52. The main composition of the particle can be the heavy element or the particle can be made of other material(s) and the heavy element incorporated by ionic or covalent attachment. Examples of the heavy element are I, Ba, La, Ce, Pr, Nd, Pm, Sm, Eu, Gd, Th, Dy, Ho, Er, Tm, Yb, Lu, W, Pt, Au, Pb or Bi. A preferred heavy element is gold, (Au). Another preferred particle is composed of iron oxide doped with a rare earth metal ion. Yet another type of preferred particle is one composed of rare earth oxide. Other desired particles for use in this invention have a dielectric core coated with a thin metallic layer and the external stimulus is an infrared laser.

Process for Treatment Using the Various Particles. The method of this invention comprises three steps. The first step is the delivery of a material that will be acted upon by the external stimulus to the synovial fluid of an affected joint. The preferred materials are particulate in nature as described above and contain at least one of magnetic particles or heavy element. Introduction of these materials into the synovial fluid is accomplished by intra-articular injection of the particles into the joint space.

The second step involves allowing the material to localize and/or concentrate in the synovial membrane. Particles of the desired size, as described earlier, are taken up by the synovial membrane. After appropriate time for uptake, the remainder of the particles may optionally be removed by infusing a saline solution into the synovial cavity and removing the fluid. This may be done multiple times until the majority of the particles remaining in the synovial cavity are associated with the synovial membrane.

The third step is to apply an external stimulus to the affected joint that has the material localized in the synovial membrane. Methods of stimulus include low energy photons or electromagnetic ionizing radiation such as gamma rays or X-radiation or near-IR laser for the particles with heavy elements or an alternating magnetic field for the particles with magnetic properties.

The invention will be further clarified by a consideration of the following examples, which are intended to be purely exemplary of the present invention.

### EXAMPLE 1

Magnetic particles (80 nanometer) consisting of an iron oxide core, and a dextran coating were derivatized via maleimide chemistry with a monoclonal antibody (ING-1). These particles were radioiodinated by placing 300 μL of the particles (20 mg/mL) into a test tube coated with IODO-GEN. I-131 (1 mCi, 20 μL) was then placed in the tube. A volume of 50 μL of a pH=7 HEPES buffer was added and the tube was allowed to stand for two minutes at room temperature. This was followed by the addition of 50 μL of tyrosine stop buffer (5 mg/500 μL of PBS). The preparation was transferred to a microcentrifuge tube with rinsing using HEPES buffer, pH 7. The microcentrifuge tube was placed on a magnet and allowed to stand at room temperature for one hour. The particles were attracted to the magnet and the liquid removed by decanting. One mL of HEPES buffer was used to wash the particles using the magnetic separation described above. This procedure was repeated until no radioactivity was detectable in the wash (about 4 times).

### EXAMPLE 2

Amine functionalized magnetic particles having a diameter range of 1-4 μm (Pierce Chemical) were labeled with I-131. This was accomplished by adding 2.0 μL of 1.0 mg/1 mL of Bolton and Hunter Reagent to a test tube coated with IODO-GEN. I-131 (0.99 mCi, 30 μL) was then placed in this tube and the tube was allowed to stand 2 minutes at room temperature. This mixture was transferred to a vial containing 0.5 mL of the magnetic particles (50 mg/mL) followed by the addition of 100 μL of 1.0M pH 8 phosphate buffer. Approximately 10 μL of 1.0M NaOH was added to achieve pH of 8. The preparation was allowed to stand at room temperature for 30 minutes; then the preparation was placed on a magnet for 30 minutes to remove the particles from suspension. The liquid was removed by decanting. 1 mL of 0.9% sodium chloride solution was used in the same manner as above to remove I-131 that was not bound to the particles. This washing procedure was repeated until no detectable radioactivity was found in the wash (about 4 times).

### EXAMPLE 3

The procedure of Example 1 was used to label amine coated 80 nanometer particles (microMod). The particles used were 19 mg/mL.

### EXAMPLE 4

A volume of 10-20 μL of the I-131 labeled ING-1 particles of Example 1 was administered into the left knee synovium of a male Sprague Dawley rat. This was accomplished using a 28 gage needle attached to a ⅓ cc syringe while the rat was under anesthesia. The rat was allowed to recover and three hours post injection the rat was euthanized and tissues and organs of interest were excised. The amount of I-131 in each was determined using a sodium iodide detector coupled to a multichannel analyzer.

Biodistribution data three hours post injection indicate more than 83% of I-131 activity to be with the subject knee joint; 7% in the urine, and about 3% in the stomach. There was no activity in the liver. This data is consistent with a large fraction of the particles remaining in the synovium. Non-synovium activity was most likely due to intracellular deiodination of the particles.

### EXAMPLE 5

Administration of I-131 labeled magnetic particles from Example 2 into the synovium of the left knee of a Sprague Dawley rat was accomplished using a ⅓ cc insulin syringe equipped with a 28 gauge needle. A volume of 5 μL of the particles was injected under anesthesia. The rat was allowed to recover and given 24 hours prior to being euthanized. Tissues and organs were excised, including the injected knee joint. The percent of injected I-131 was determined in each sample using a sodium iodide gamma detector.

Synovial membrane sections were excised and washed to remove non-bound particles. Microscopic examination of the synovial membrane indicated intracellular incorporation of the particles. The biodistribution of the radioisotope was consistent with almost all the particles remaining in the synovium.

### EXAMPLE 6

A volume of 5.0 μL of the I-131 labeled 80 nm magnetic particles of Example 3 were administered into the left knee synovium of a Sprague Dawley rat as described in Example 4.

Twenty four hours after administration the rat was sacrificed and tissues and organs removed. Microscopic examination of the excised synovial membrane showed a significant number of particles embedded in the synovial membrane. The biodistribution of the radioisotope was consistent with almost all the particles remaining in the synovium.

These results show that for all examples the particles remained mainly in the desired synovial membrane.

Although the invention has been described with reference to its preferred embodiments, those of ordinary skill in the art may, upon reading and understanding this disclosure, appreciate changes and modifications which may be made which do not depart from the scope and spirit of the invention as described above or claimed hereafter.

1. A method of performing synovectomy in an affected joint comprising:
(a) administering to the affected joint an effective amount of a material that becomes associated with the synovial membrane;
(b) allowing the material to localize and/or concentrate in the synovial membrane; and
(c) applying an external stimulus to the affected joint which interacts with the material, thus delivering a therapeutic dose to ablate the synovial membrane.

2. The method of claim 1 wherein the material administered consists of particles.

3. The method of claim 2 wherein the particles are between 15 nanometers and 15 microns.

4. The method of claim 3 wherein the particles are between 25 nanometers and 5 microns.

5. The method of claim 3 wherein the particles are between 50 nanometers and 3 microns.

6. The method of any one of claims 1 to 5 wherein the material consists of magnetic particles and the external stimulus is an alternating magnetic field.

7. The method of claim 6 wherein the magnetic particles contain iron oxide or hydroxide.

8. The method of claim 7 wherein the magnetic particles have a surface coating.

9. The method of claim 8 wherein the surface coating is cationic at physiological pH.

10. The method of any one of claims 1 to 5 wherein the material or particle contains a heavy element having an atomic number greater than 37, and the external stimulus is low energy photons or electromagnetic ionizing radiation.

11. The method of claim 10 wherein the low-energy photons or electromagnetic ionizing radiation are gamma rays or X-rays.

12. The method of claim 10 wherein the heavy element has an atomic number greater than 52.

13. The method of claim 12 wherein the heavy element is I, Ba, La, Ce, Pr, Nd, Pm, Sm, Eu, Gd, Th, Dy, Ho, Er, Tm, Yb, Lu, W, Pt, Au, Pb or Bi.

14. The method of claim 13 where the heavy element is Au.

15. The method of any one of claims 1 to 5 wherein the material contains particles composed of a dielectric core coated with a thin metallic layer and the external stimulus is an infrared laser.

16. The method of any one of claims 1 to 5 wherein the material or particle is administered as a pharmaceutically-acceptable formulation.

17. A method of treating chronic synovitis in a patient comprising using the method of any one of claims 1 to 16.

18. The method of claim 17 wherein the disease causing the chronic synovitis is arthritis.

19. The method of claim 18 wherein the arthritis is rheumatoid arthritis or psoriatic arthritis.
